(19) United States
(12) Patent Application Publication
(10) Pub. No.: US 2025/0182841 A1
(43) Pub. Date: Jun. 5, 2025

(54) MULTIMODAL GRAPH

(71) Applicant: CANON MEDICAL SYSTEMS CORPORATION, Otawara-shi (JP)

(72) Inventors: Simon FISHER, Edinburgh (GB); Russell HUNG, Edinburgh (GB)

(73) Assignee: CANON MEDICAL SYSTEMS CORPORATION, Otawara-shi (JP)

(21) Appl. No.: 18/527,460

(22) Filed: Dec. 4, 2023

(51) Int. Cl. G16B 15/30; G16B 25/10; G16H 20/10

(52) U.S. Cl. CPC G16B 15/30; G16B 25/10; G16B 45/00; G16H 20/10

(57) ABSTRACT

A medical information processing apparatus comprising a processing circuitry configured to: receive drug target information based on a medical condition, the drug target information comprising at least one target biomolecule each associated with a respective drug suitable for the treatment of said medical condition; receive protein-protein interaction information based on domain knowledge, receive multimodal omics data for at least one subject; construct a graph based upon the drug target information, the protein-protein interaction information and the multimodal omics data; and generate, for each of the at least one drug, a score for each of the at least one subject based on the graph and the multimodal omics data. For each of the at least one subject, the drugs may be ranked based on their respective generated score.

### FIELD

The present invention relates to the field of integrating multimodal omics data and domain knowledge, in particular for precision medicine applications.

### BACKGROUND

Precision medicine is the process by which patient-specific characteristics (usually genetic data) are used to inform clinical decision making. In the field of pharmacogenomics, a canonical precision medicine pipeline begins by identifying mutations in a patient by performing a genome-wide association (GWAS), associating any of the identified mutations with a drug response, and then subsequently changing the drug treatment of the patient based on the presence of any mutations.

Many experts once regarded precision pharmacogenomics as heralding a new wave of medicine, whereby the patient was at the centre of decision making, and patient mutational data would underpin virtually all medicine. Some may now regard GWAS-based precision medicine as generally failing to deliver, with very few actionable protocols existing, especially outside cancer. In one study by Massard et al. it was found that the administration of targeted therapies based on genomic analyses of tumour biopsies improved the outcomes in a subset of patients with hard-to-treat cancers (Cancer discovery 7.6 (2017): 586-595). However, only 7% of the successfully screened patients had an improvement in clinical outcome using this approach. It is therefore questionable whether the point-mutation precision medicine hypothesis, which considers the role of individual gene mutations, is capturing the full picture to enable the effective administration of targeted therapies.

Multi-omics approaches may offer a way to improve the efficacy of precision pharmacogenomics approaches. Current protocols currently only integrate single mutations, or more rarely, tumour mutational burden. They therefore do not take into account the useful information from other omics modalities, which can be encoded as a gene network or an integrated multi-omics network. Increasingly, especially the in context of cancer, patient data belonging to these additional modalities is available, as is the domain knowledge for forming patient graphs. The lack of consideration of this additional information may be impacting the success of precision medicine approaches.

### BRIEF DESCRIPTION OF THE DRAWINGS

Embodiments are now described, by way of non-limiting example, and are illustrated in the following figures, in which:

FIG. 1 is a schematic diagram of an apparatus according to an embodiment;

FIG. 2 is a schematic of a method according to an embodiment;

FIG. 3 is a flow chart of a method according to an embodiment;

FIG. 4 is a schematic of a sub-graph showing the interactions between a drug and its drug gene target, and the interactions of proteins with that drug gene target.

FIG. 5 is an illustration of a plurality of sub-graphs as shown in FIG. 4, where each sub-graph corresponds to a drug suitable for the treatment of a medical condition. A score table shows a score for each respective drug based on multimodal omics data assigned to the network.

### DESCRIPTION

Certain embodiments provide a medical information processing apparatus comprising a processing circuitry configured to: receive drug target information based on a medical condition, the drug target information comprising at least one target biomolecule, each of the at least one target biomolecule associated with a respective drug suitable for the treatment of said medical condition; receive protein-protein interaction information based on domain knowledge, receive multimodal omics data for at least one subject; construct a graph based upon the drug target information, the protein-protein interaction information and the multimodal omics data; and generate, for each of the at least one drug, a score for each of the at least one subject based on graph and the multimodal omics data.

Certain embodiments provide a medical information processing method, the method comprising: receiving drug target information based on a medical condition, the drug target information comprising at least one target biomolecule, each of the at least one target biomolecule associated with a respective drug suitable for the treatment of said medical condition; receiving protein-protein interaction information based on domain knowledge, receiving multimodal omics data for at least one subject; constructing a graph based upon the drug target information, the protein-protein interaction information and the multimodal omics data; and generating, for each of the at least one drug, a score for each of the at least one subject based on the graph and the multimodal omics data.

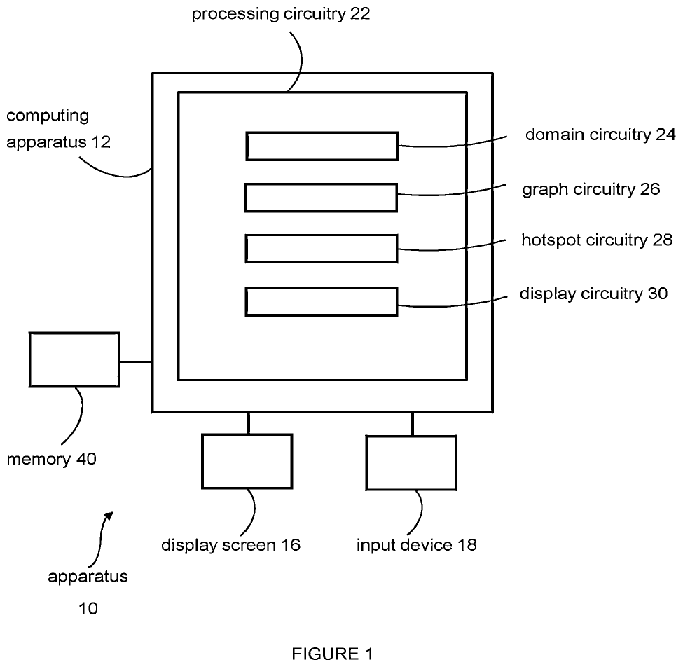

An apparatus 10 according to an embodiment is illustrated schematically in FIG. 1. The apparatus 10 may also be referred to as a medical information processing apparatus. The apparatus 10 is configured to process domain knowledge 50 and multimodal omics data 70. The apparatus 10 is further configured to display an image based on the domain knowledge 50 and multimodal omics data 70.

The domain knowledge 50 comprises information relating to drug indications, drug-target interactions, and protein-protein interactions, as well as gene expression datasets and gene variant datasets, all of which may be either stored in the memory or accessible online.

The multimodal omics data 70 may correspond to omics data from one subject or any number of subjects. The multimodal omics data 70 may comprise gene expression data 71 and gene variant data 73.

In other embodiments, the apparatus 10 may be configured to process any appropriate data.

The apparatus 10 comprises a computing apparatus 12, which in this case is a personal computer (PC) or workstation. The computing apparatus 12 is connected to a display screen 16 or other display device, and an input device or devices 18, such as a computer keyboard and mouse. The computing apparatus 22 receives data from memory 40, which may also be referred to as a data store or storage. In alternative embodiments, computing apparatus 12 receives data from one or more further data stores (not shown) instead of or in addition to memory 40. For example, the computing apparatus 12 may receive data from one or more remote data stores (not shown), which may comprise cloud-based storage.

The memory 40 stores multimodal omics data. The memory 40 further stores domain knowledge.

In other embodiments, the multimodal omics data and domain knowledge may be stored in another suitable memory, for example in another apparatus or in a cloud-based memory. The omics data can be stored in any file format suitable for storing text-based data such as TSV, CSV, XLS, XML or VCF and MAF for mutational data. The domain knowledge may be stored in any suitable format such as a database.

Computing apparatus 12 comprises a processing circuitry 22 for processing data. The processing circuitry 22 comprises a central processing unit (CPU) and Graphical Processing Unit (GPU). The processing circuitry 22 provides a processing resource for automatically or semi-automatically processing omics datasets.

The processing circuitry 22 includes a domain circuitry 24 which obtains domain knowledge, a graph circuitry 26 which constructs a graph based on the domain knowledge, a hotspot circuitry 28 which adds data to the graph based on multimodal omics data and calculates a score for one or more drugs based on graph for each subject, and a display circuitry 30 which displays the scores and graph.

In the present embodiment, the circuitries 24, 26, 28 and 30 are each implemented in the CPU and/or GPU by means of a computer program having computer-readable instructions that are executable to perform the method of the embodiment. In other embodiments, the circuitries may be implemented as one or more ASICs (application specific integrated circuits) or FPGAs (field programmable gate arrays).

The computing apparatus 12 also includes a hard drive and other components of a PC including RAM, ROM, a data bus, an operating system including various device drivers, and hardware devices including a graphics card. Such components are not shown in FIG. 1 for clarity.

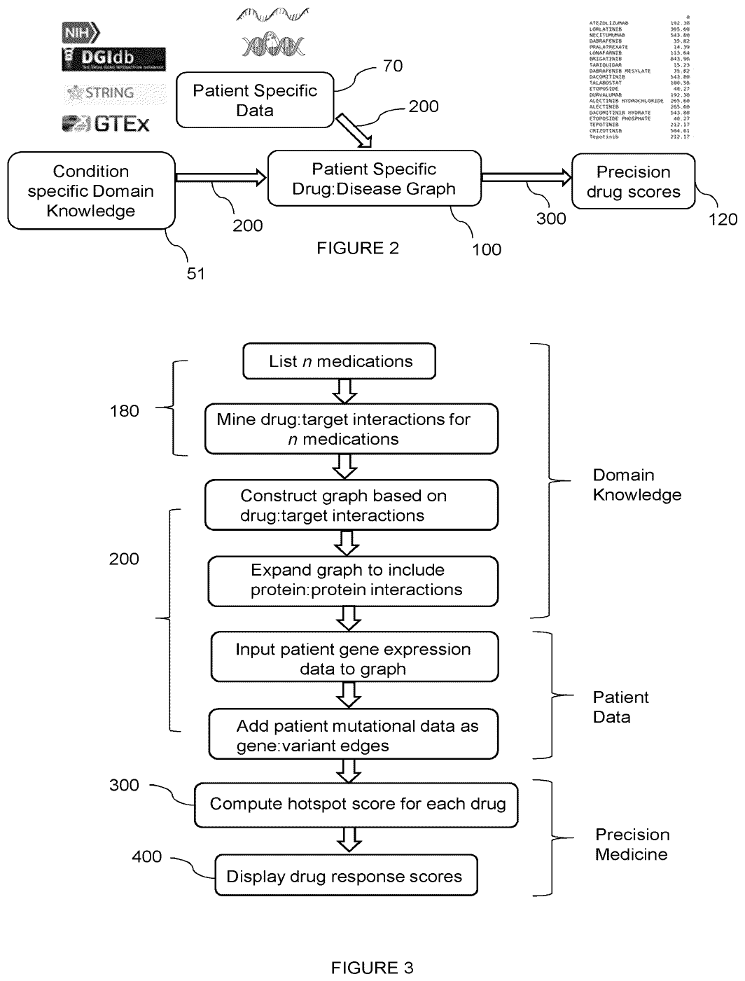

The processing circuitry 22 of FIG. 1 is configured to perform a method in accordance with FIG. 2. FIG. 2 is a flow chart illustrating in overview a method for generating precision drug stores.

At stage 200, the graph circuitry 26 receives a subset of the domain knowledge 50 which is specific to a medical condition, referred to as condition-specific domain knowledge 51. The condition-specific domain knowledge 51 comprises a list of drugs 52 which are suitable for the treatment of the medical condition and drug-target information 63, which maps a respective biomolecule 53 to each of the respective drugs 52. Each of the biomolecules 53 correspond to a primary target of a respective drug of the list of drugs 52 . . . . Each of the target biomolecules 53 may be a gene, RNA or protein respectively.

The graph circuitry 26 constructs a graph 100 based on the condition-specific domain knowledge 51. The graph circuitry 26 then obtains protein-protein information 64 which maps any number of proteins 54 to each of the respective biomolecules 53 based on interactions between proteins 54 and the proteins that correspond to said biomolecules 53.

The graph circuitry 26 then expands the graph 100 based on the protein-protein information 64.

The graph circuitry 26 then receives multimodal omics data 70 comprising gene expression data 71. The gene expression data 71 comprises gene expression values 74 for one or more subjects that are affected by the medical condition. The gene expression values 74 are normalized relative to a plurality of subjects who are categorized as suffering from the same medical condition as the subjects in the multimodal omics data 70. The gene expression values 74 may be absolute values of z-score normalized expression values that are normalized relative to a cohort of subjects.

The multimodal omics data 70 may further comprise gene variant data 73 for one or more subjects indicating the presence of gene variants.

The graph circuitry 26 inputs values to the graph 100 based on normalized gene expression values 74 and the gene variant data 73.

At stage 300, the hotspot circuitry 26 calculates a set of scores 120 comprising a score 35 for each of the drugs 52 based on the graph 100. If the multimodal omics data 70 relates more than one subject, the set of scores comprise a score for each of the drugs 52 for each respective subject. The score may act as a proxy for the likelihood of local patient-specific entities affecting drug response. For example, the drug response in a subject receiving a highly scoring drug may be stronger or weaker than expected.

Turning to FIG. 3, the method for generating precision drug scores will now be explained in further detail.

At stage 180, a user identifies a medical condition. The medical condition may be any medical condition for which drug treatment is available. The medical condition may be a condition affecting one or more subjects in a clinical setting. The medical condition may be the focus of a clinical trial for which a new drug is being developed. The medical condition may be the focus of research and development processes in a pharmaceutical company.

The domain knowledge 50 is searched based on the medical condition to identify the condition-specific domain knowledge 51 comprising a list of drugs 52 and drug-target information 63. The list of drugs 52 may be obtained by searching a database such as the National Institute of Health website, which lists the drugs suitable for the treatment of various medical conditions. The drug-target information can be obtained from databases such as the DGIdb (Drug Gene Interaction database). The drug-target information 63 comprises a set of biomolecules 53 that are each mapped to a respective drug 52 based on the interactions between the drug and that biomolecule 53.

The condition specific domain knowledge 51 may be obtained by the user searching the one or more relevant databases or it may be obtained automatically by the domain circuitry 24.

At the end of stage 180, the condition specific domain knowledge 51 is output to the graph circuitry 26.

At stage 200, the graph circuitry 26 receives the condition-specific domain knowledge 51.

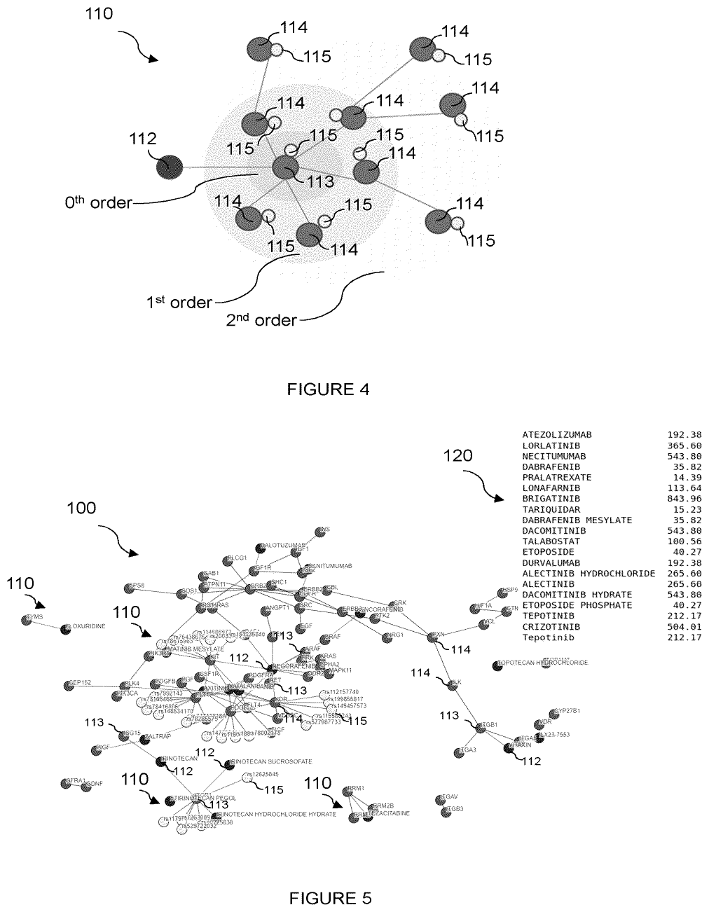

The graph circuitry 26 constructs a graph 100 based on the condition-specific domain knowledge 51. A graph comprises a set of nodes which are connected by one or more edges. The graph 100 comprises a set of sub-graphs 110 for each of the respective drugs 52. FIG. 4 illustrates a schematic of a sub-graph 110. Each of the sub-graphs 110 have a starting node 112 which represents a respective drug 52. Each of the starting nodes 112 are connected to a respective node 113 representing a biomolecule 53 according to the drug-target information 63. Although a drug may act on more than one gene or gene product (i.e. RNA or protein), in the present embodiment, a starting node 112 is connected to only one node 113 representing a biomolecule which is a primary target of the drug represented by node 112.

Based on the biomolecules 53, the graph circuitry 26 obtains protein-protein information 64 from the domain knowledge 50. Protein-protein information 64 is accessible from databases such as STRING (Search Tool for the Retrieval of Interacting Genes/Proteins). The protein-protein information 64 maps any number of proteins 54 to a biomolecule 53 based on the knowledge of interactions between a protein 54 and the protein corresponding to biomolecule 53. For example, if a target biomolecule 53 is a protein, the protein-protein information 64 comprises information relating to the interaction between the protein 53 and one or more proteins 54. If a target biomolecule 53 is an RNA or gene, the protein-protein information 64 comprises information relating to the interaction between a protein that is the product of the RNA or gene 53 and one or more proteins 54.

The graph circuitry 26 expands the graph 100 based on the protein-protein information 64. The graph 100 is expanded by connecting each of the nodes 113 to one or more nodes 114 representing a protein 54 in accordance with the protein-protein interaction information 64. Since proteins may interact with more than one other protein, each biomolecule 53 may be mapped to more than one protein. 54 Accordingly, a node 113 may be mapped to more than one node 114. It is also possible that some biomolecules 53 will not be mapped to any proteins 54. Accordingly, it is possible that some nodes 113 are not mapped to any nodes 114.

The protein-protein information 64 may correspond to any number of degrees of connections. Accordingly, the graph 100 may be expanded by one order if the protein-protein information 64 comprises first degree connections, by two orders if the protein-protein information comprises first and second degree connections, and by three orders if the protein-protein information comprises first, second and third degree connections, etc. The example shown in FIG. 4 shows the sub-graph 110 expanded by nodes 114 that are either one or two orders from node 113.

In other embodiments, it is envisaged that instead of the protein-protein information 64 being obtained by the graph circuitry 26 at stage 200, it is alternatively obtained by the domain circuitry 24 at stage 180.

The graph circuitry 26 receives multimodal omics data 70 for one or more subjects. The one or more subjects may be patients in a clinical setting, participants in scientific studies, donors for biobanks, or any combination of these. The multimodal omics data may be omics data from measurements performed on a tissue sample, such as tumor sample, blood sample, or cells obtained from a primary culture of a tissue biopsy.

The multimodal omics data 70 comprises gene expression data 71 and gene variant data 73 for one or more subjects affected by the medical condition. Gene expression data 71, also known as transcriptomic data, comprises gene expression values 72 for a plurality of genes for each of the one or more subjects. The gene expression values 72 may be detected using any suitable technique for detecting RNA such as microarray, qRT-PCR or RNA-Seq. Gene variant data 73 relates to the presence of one or more changes in genetic sequence. If the multimodal omics data 70 is obtained based on tumor samples, the gene variant data 73 may comprise the tumor mutational burden at specific loci in the genome, which corresponds to the number of cancerous mutations at respective genes or other DNA segments in the genome. In addition to this, or alternatively, the gene variant data may relate to individual variants from either somatic or germline origin, including SNPs and Insertions/Deletions.

The graph circuitry 26 normalizes the gene expression values 72 relative to a plurality of subjects that are classified as being affected by the same medical condition as the one or more subjects in the multimodal omics data 70 and then takes the absolute of the normalized values to result in normalized gene expression values 74. In addition to suffering from the same medical condition, the subjects may also suffer similar side effects based on a treatment for the medical condition. The normalized gene expression values 74 therefore reflect gene expression levels in relation to an expected, or average, disease state.

If the gene expression data 71 relates to more than one subject, the normalization may be performed based on gene expression values 72. Alternatively, the normalization may be performed using gene expression values from a separate dataset stored in the memory 40 or any other suitable data store. The separate dataset may be part of the domain knowledge 50 or may be cohort data to which a clinician has access. Suitable datasets may be obtained using databases such as TCGa (The Cancer Genome Atlas Program) or the GTEx.

The normalization may be a z-score normalization which normalises each expression value x based on the function z=(x−μ)/σ, where u is the mean expression value for that gene across all subjects, and σ is the standard deviation for that gene across all subjects. Alternatively, any other suitable normalization method may be used, such as mix-max scaling, or scaling to a range.

In the present embodiment, the gene expression values 72 are normalized by the graph circuitry 26 to produce normalized gene expression values 74. In other embodiments, the gene expression data 71 may already comprise normalized gene expression values 74.

The graph circuitry 26 assigns values to the nodes 113, 114 of the graph 100 based on the normalized gene expression values 74. For each of the nodes 113, 114, the corresponding normalized gene expression value for the biomolecule or protein represented by the respective nodes 113, 114 is assigned. If the gene expression data 71 relates to more than one subject, the normalized gene expression values 74 will be assigned to a node 113, 114 as a vector of gene expression values.

The graph circuitry 26 then further expands the graph 116 based on gene variant data 73. The graph 100 is further expanded by connecting a node 113, 114 to a node 115 by an edge representing a gene variant of the gene or protein represented by node 113, 114. Each of the gene variant edges represent a respective gene variant. Values are assigned to the nodes 115 based on the presence or absence of the gene variant for the one or more subjects in the gene variant data 73. If the gene variant data 73 relates to more than one subject, values are assigned to the nodes 115 as a vector. A value of one is assigned to a node 115 if a subject has the gene variant represented by the corresponding gene variant edge and a value of zero is assigned to the node 115 if a subject does not have the corresponding gene variant represented by the corresponding gene variant edge. FIG. 4 schematically shows each of the nodes 113, 114 connected to a respective node 115 . . . . Although FIG. 4 shows only one node 115 connected to any node 113, 114, in a real use-case it is expected that there would be many more nodes 115 connected to a respective node 113, 114.

At the end of stage 200, the graph circuitry outputs the graph 100 to the hotspot circuitry 26.

At stage 300, the hotspot circuitry 26 receives the graph 100.

The hotspot circuitry 26 calculates a score for each of the drugs 52 based on the graph 100 to produce a set of scores 120. A score for each drug 52 is determined by considering the values assigned values to the nodes 113, 114, 115 of the sub-graph 110. The score is a function of X, where X are the normalized gene expression values 74 and mutation counts assigned to nodes 113, 114, 115 weighted by the distance of the nodes 113, 114, 115 from node 113.

When calculating the score, each of nodes 115 are considered to have a degree of separation relative to node 113 as the respective node 113, 114 that it is connected to. For instance, and as shown in FIG. 4, nodes 115 connected to node 113 are 0th order nodes, nodes 115 connected to nodes 114 which are one degree of separation from nodes 113 are 1st order nodes, nodes 115 connected to nodes 114 which are two degrees of separation from nodes 113 are 2nd order nodes, etc.

The nodes 113, 114, 115 that are located up to two degrees of separation from a node 113 can be considered the local neighbourhood of a drug and it is with these nodes that the score is calculated.

The score is calculated by summing the following values:
For 0th order nodes 113, 115:
   absolute normalized gene expression value and mutation count
For 1st order nodes 114, 115:
   (absolute normalized gene expression value and associated mutation count)*(⅔)
For 2nd order nodes 114, 115:
   (absolute normalized gene expression value and associated mutation count)*(⅓).

Nodes 114, 115 that are three orders or more away from nodes 113 are not included in the score calculation. If the multimodal omics data 70 comprises only one subject, the score for each drug is a single value. If the multimodal omics data 70 comprises more than one subject, the score for each drug is a vector of values.

In other embodiments, any other suitable function may be used to calculate the set of hotspot scores 120. For example, any function of the normalized gene expression values and mutations assigned to nodes 113, 114, 115, weighted by the distance of the nodes 113, 114, 115 from node 113, may be used.

The hotspot circuitry 26 ranks the drugs 52 for each of the one or more subjects based on the scores 120.

At the end of stage 300, the hotspot circuitry 26 outputs the graph 100 and scores 120 to the display circuitry 28.

At stage 400, the display circuitry 28 displays the scores 120 and the graph 100. In some embodiments, the highest ranked drugs are highlighted as possible prescribing options.

FIG. 5 illustrates a graph 100 comprising a plurality of subgraphs 110 each corresponding to a drug suitable for the treatment of colorectal cancer and the calculated scores 120. Reference numerals have been provided for only a subset of features in the graph 100. It can be seen that some of the subgraphs 110 are connected to other subgraphs 110 due to some of nodes 113, 114 being common nodes to respective pairs of subgraphs 110. As can be seen in FIG. 5, nodes 112, 113, 114 and gene variant edges are annotated with the names of the corresponding drug, gene, protein or gene variant.

In further embodiments, as shown in FIG. 5, further targets of a drug extending beyond the primary targets, may be considered. For example, a drug 52 may also be associated with one or more secondary targets, or off-targets, of the drug. In these embodiments, a node 112 may be connected to more than one node 113.

In further embodiments, the method described herein may be expanded to include other sources of data. For example, the multimodal omics data 70 may include mass spectrometry data in addition to, or as an alternative to the gene expression data 71. The mass spectrometry data may comprise for each subject protein abundance values for a plurality of proteins obtained by performing quantitative high-throughput mass spectrometry on a tissue sample from each respective subject. As gene expression does not always correlate to the abundance of proteins, the use of mass spectrometry data to calculate the hot spot scores 120 may result in more relevant hot spot scores.

In addition, in further embodiments, the graph 100 may be expanded to include further nodes and edges representing other types of omics data relevant to a subject. For example, nodes 113, 114 may be connected to nodes corresponding to epigenetic information, such as DNA methylation, and values may be assigned to these nodes according to the degree of methylation. Further nodes may be added corresponding to single cell data. The graph 100 may be further expanded based on health economics information. For example, nodes may be added to each of the subgraphs 110 assigned with values corresponding to the economic cost of making the respective drug available.

In other embodiments, the method described herein may be expanded to incorporate in vitro pharmacological data, such as IC50 measurements indicating how much of a drug is needed to inhibit a biological process or component by 50%, or EC50 measurements, which indicate the amount of a drug needed to produce half the maximal response. IC50/EC50 values for one or more of the drugs 52 for respective one or more samples can be obtained based on in vitro dose-response experiments performed on one or more samples. In parallel, sample-specific omics data such as gene expression and gene variant data may be obtained for the one or more samples on which the dose-response experiments are performed. Using the methods disclosed herein, a set of scores 120 for each sample comprising a respective score for each drug may be calculated based on the corresponding omics data for that sample. Based on this information, a model for each drug may be constructed to predict a relationship between a IC50/EC50 of the drug and a hotspot score. Each of the models may be used to provide a subject-specific IC50/EC50 prediction for one or more drugs by inputting the drug specific score for that subject into the model using the multimodal omics data 70.

Advantageously, the systems and methods described herein provide a precision medicine approach for evaluating potential drug responses in a subject based on subject-specific omics data and domain knowledge. The approach described herein is particularly suitable for medical conditions such a cancer because a tumour sample can be used to obtain the omics data, but the approach may also be applied to other medical conditions, such as cardiovascular disease, for which a relevant tissue sample may be blood.

The systems and methods described herein can be used as a clinical decision support in the context of choosing pharmacological interventions for patients. For example, if the scores 120 indicate that for certain drugs there may be a high likelihood of a local gene/protein network being strongly affected in a patient, the clinician may be motivated to consider the implications of this when prescribing. In situations where the scores for two drugs for a patient are tied based on omics data, health economics data may be incorporated into the graph as an aid to clinical decision making.

The systems and methods described herein may also be used as an aid to clinical research for identifying novel precision medicine hypotheses. By retroactively examining drug response datasets, these scores could be used to provide precision medicine insights. For example, if a cohort of patients stratified by experiencing a certain side-effect to a specific drug were found to have high hotspot scores for that drug compared to other patients not experiencing the side-effect, the individual variants on the nodes in the hotspot region could be examined and/or the gene expression values could be investigated to see if certain gene variants and/or gene expression values are responsible for the high hotspot score. The systems and methods described herein may also be applied to clinical research to identify cohorts of patients suitable for clinical trials. Subjects that have similar scores be grouped together as a cohort of patients.

The systems and methods described herein may also be used in clinical research in order to highlight possible precision biological effects of new compounds during R&D. For example, the targets of novel drugs can be screened by inputting the target as drug target 52 and outputting scores 120 to see predicted subject-specific responses to the drug.

Certain embodiments provide a method for calculating a response score derived from a graph consisting of drug: gene, gene: gene and gene: variant edges which is used to incorporate multimodal patient data. The graph is initially populated by a plurality of drugs used to treat a condition, with initial edges consisting of drug: gene interactions, mined from domain knowledge. The graph is subsequently expanded to capture the gene-gene interactions of these neighbourhoods. The patient-specific gene expression, z-scored against the expected expression is used to annotate gene nodes. Patient-specific mutational data is incorporated into the graph via gene: mutation edges. An arithmetic operation is performed on local neighbourhoods, normalised to degree, to create a weighted score of possible drug responses specific to individual patients

The method may further comprise the incorporation of further multi-omics modalities such as DNA methylation, single-cell sequencing and mass spectrometry.

The method may further comprise the incorporate of health economics data.

The scores may be based on any other arithmetic or other operation using the presence of mutations and aberrations in gene expression alongside degree of proximity to drug target.

The method may further comprise the incorporation of retrospective datasets on drug response to allow for the modelling of IC50 or EC50 scores as opposed to a simple value.

Certain embodiments provide a medical data processing apparatus comprising processing circuitry configured to: receive a graph configured by nodes and edges, wherein the nodes comprising drug, a variant gene, and gene related to at least one of the drug or the variant gene and the edges comprising an interaction between the nodes, wherein the drug and the variant gene are correlated by related genes in the graph, receive variant gene information related to a subject, determine a first node corresponding to the variant gene information on the graph, and determine, based on the first node, a second node corresponding to drug related to the variant gene information on the graph.

Whilst particular circuitries have been described herein, in alternative embodiments functionality of one or more of these circuitries can be provided by a single processing resource or other component, or functionality provided by a single circuitry can be provided by two or more processing resources or other components in combination. Reference to a single circuitry encompasses multiple components providing the functionality of that circuitry, whether or not such components are remote from one another, and reference to multiple circuitries encompasses a single component providing the functionality of those circuitries.

Whilst certain embodiments have been described, these embodiments have been presented by way of example only, and are not intended to limit the scope of the invention. Indeed the novel methods and systems described herein may be embodied in a variety of other forms. Furthermore, various omissions, substitutions and changes in the form of the methods and systems described herein may be made without departing from the spirit of the invention. The accompanying claims and their equivalents are intended to cover such forms and modifications as would fall within the scope of the invention.

1. A medical information processing apparatus comprising a processing circuitry configured to:
receive drug target information based on a medical condition, the drug target information comprising at least one target biomolecule, each of the at least one target biomolecule associated with a respective drug suitable for the treatment of said medical condition;
receive protein-protein interaction information based on domain knowledge,
receive multimodal omics data for at least one subject;
construct a graph based upon the drug target information, the protein-protein interaction information and the multimodal omics data; and
generate, for each of the at least one drug, a score for each of the at least one subject based on the graph and the multimodal omics data.

2. The medical information processing apparatus of claim 1, wherein the processing circuitry is configured to construct the graph by being further configured to, for each of the at least one drug:
add a node to the graph corresponding to the respective target biomolecule;
expand the graph by connecting, with an edge, the node corresponding to the respective target biomolecule to at least one additional node corresponding to a gene or protein, wherein the edge corresponds to a first or second degree connection between the target biomolecule and the gene or protein based upon the protein-protein interaction information; and
further expand the graph by connecting, with an edge, one or more nodes of the graph to an additional node corresponding to a biomolecule based upon the multimodal omics data.

3. The medical information processing apparatus of claim 2, wherein the multimodal omics data comprises gene expression data, the gene expression data comprising gene expression levels for a plurality of genes normalized with respect to a plurality of subjects.

4. The medical information processing apparatus of claim 3, wherein said score is generated based upon assigning the gene expression levels to each of the nodes of the graph corresponding to a target biomolecule and gene or protein.

5. The medical information processing apparatus of claim 4, wherein the contribution of a gene expression level assigned to a node to the score is weighted based upon the degree of separation of the node from the node representing the target biomolecule.

6. The medical information processing apparatus of claim 5, wherein the weighting is inversely proportional to the degree of separation.

7. The medical information processing apparatus of claim 4, wherein the score is calculated based on absolute gene expression levels.

8. The medical information processing apparatus of claim 2, wherein the multimodal omics data comprises gene variant information indicating the presence of gene mutations associated with the at least one target biomolecule and/or at least one gene or protein represented by the graph; wherein the graph is further expanded based upon the gene variant information.

9. The medical information processing apparatus of claim 1, wherein the graph comprises:
at least one starting node representing a respective drug;
at least one 0^{th} order node representing a respective target biomolecule, wherein each of the 0^{th} order nodes are connected to a respective starting node by an edge based on the drug target information;
at least one 1^{st} order node representing a respective gene or protein, wherein each of the 1^{st} order nodes are connected to a 0^{th} order node by an edge based on the protein-protein interaction information;
at least one 2^{nd} order node representing a respective gene protein, wherein each of the 2^{nd} order nodes are connected to a 1^{st} order node by an edge based on the protein-protein interaction information;
any number of nodes representing a gene variant connected to a respective 0^{th} order, 1^{st} order or 2^{nd} order node by a gene variant edge based on the gene variant information;
wherein a first value is assigned to each of the 0^{th} order, 1^{st} order and 2^{nd} order nodes based on the gene expression levels;
wherein a second value is assigned to each of the 0^{th} order, 1^{st} order and 2^{nd} order nodes based on the presence of a gene variant; and
wherein the score for each drug for each subject is calculated by summing the values for said subject assigned to the nodes originating from the starting node representing said drug, wherein the values are weighted based on the distance of each corresponding node from the respective 0^{th} order node.

10. The medical information processing apparatus of claim 1, wherein the multimodal omics data is normalized with respect to a plurality of subjects stratified by the medical condition and/or a side effect from a treatment for the medical condition.

11. The medical information processing apparatus of claim 1, wherein each of the target biomolecules correspond to a gene, RNA or protein that is targeted and/or modulated by a respective drug.

12. The medical information processing apparatus of claim 1, wherein each of the target biomolecules are a primary target of a respective drug.

13. The medical information processing apparatus of claim 1, wherein the multimodal omics data further comprises at least one of DNA methylation, single-cell sequencing and mass spectrometry for at least one subject.

14. The medical information processing apparatus of claim 1, wherein the processing circuitry is further configured to:
receive health economics data relating to the at least one drug
wherein said score is further based on the health economics data.

15. The medical information processing apparatus of claim 1, wherein the processing circuitry is further configured to:
receive IC50 or other response information for at least one drug and associated omics data for a plurality of samples;
generate, for each of the at least one drug, a score for each of the plurality of samples based on a graph constructed based upon the target biomolecule associated with said drug, the protein-protein interaction information and the omics data for said sample;
construct a mapping for each of the at least one drug defining a relationship between a score and the associated IC50 or other response information;
predict a response metric for each of the at least one subject for each of the at least one drug based on the respective score for that subject and the mapping.

16. The medical information processing apparatus of claim 1, wherein the drug target information relates to a plurality of drugs, and wherein the processing circuitry is further configured to:
for each of the at least one subject, rank each of the plurality of drugs based on the score generated for the respective drug.

17. The medical information processing apparatus of claim 1, wherein the processing circuitry is further configured to:
select, based on the score for each drug for at least one subject, a suitable line of treatment for the respective subject.

18. The medical information processing apparatus of claim 1, further comprising a display circuitry configured to:
display the graph and the at least one generated score.

19. A medical information processing method, the method comprising:
receiving drug target information based on a medical condition, the drug target information comprising at least one target biomolecule, each of the at least one target biomolecule associated with a respective drug suitable for the treatment of said medical condition;
receiving protein-protein interaction information based on domain knowledge,
receiving multimodal omics data for at least one subject;
constructing a graph based upon the drug target information, the protein-protein interaction information and the multimodal omics data; and
generating, for each of the at least one drug, a score for each of the at least one subject based on the graph and the multimodal omics data.
